(19) United States
(12) Patent Application Publication
(10) Pub. No.: US 2026/0192008 A1
(43) Pub. Date: Jul. 9, 2026

(54) Safety-Controlled Ozone Generator with Motion Detection and Configurable Parameters

(71) Applicant: Richard Taylor, Dayton, OH (US)

(72) Inventor: Richard Taylor, Dayton, OH (US)

(21) Appl. No.: 19/013,001

(22) Filed: Jan. 8, 2025

(51) Int. Cl. A61L 9/12; A61L 101/02

(52) U.S. Cl. CPC A61L 9/12; A61L 2101/02; A61L 2209/111

(57) ABSTRACT

An ozone generator system includes a motion detection module with at least one motion sensor configured to detect motion within a monitored area. A control module is operatively coupled to the motion detection module and an ozone generation module. The control module initiates ozone generation in response to a start command, terminates ozone generation upon detection of motion, and prevents further ozone generation until a manual restart command is received. An alert module, operatively coupled to the control module, includes an audible indicator and a visual indicator to provide real-time alerts in response to motion detection. An interface allows adjustment of at least one operational parameter of the motion detection module, including motion detection sensitivity. The motion detection module further includes a plurality of motion sensors arranged to monitor distinct zones, with the control module managing ozone generation based on signals from any of the monitored zones.

### FIELD OF INVENTION

The present invention relates to ozone generator systems, specifically to systems incorporating motion detection, safety alerts, and configurable control parameters to enhance operational safety and flexibility in various environments.

### BACKGROUND

Ozone generators are extensively utilized across residential, commercial, and industrial settings for air purification, odor removal, and disinfection. These devices generate ozone gas, which is highly effective in neutralizing odors, eliminating bacteria, and breaking down pollutants. Despite their utility, ozone generators present significant safety risks when improperly used, particularly concerning human exposure to ozone. Prolonged or excessive exposure to ozone can cause respiratory irritation and other adverse health effects, necessitating stringent precautions to ensure safe operation. Current systems, however, often fall short in addressing these risks comprehensively, leaving users vulnerable to accidental ozone exposure.

Traditional ozone generators rely predominantly on passive safety mechanisms, such as printed warnings or instructional labels that advise users to vacate the area during operation. These passive measures depend entirely on user compliance and vigilance, which are often inconsistent, especially in shared or dynamic spaces. In environments with multiple occupants, transient traffic, or pets, there is a significant risk of accidental exposure, as individuals may unknowingly enter an area during ozone treatment. Furthermore, such systems lack safeguards to prevent inadvertent reactivation of ozone production, for instance, after power interruptions or user errors, exacerbating the potential for harm.

Efforts to enhance safety in ozone generators have introduced features such as timers and remote control operation, allowing users to schedule or manage ozone production from a distance. While these advancements offer improved convenience, they fail to address real-time safety concerns. For instance, a timer cannot adapt to changing conditions, such as a person entering the area during an active ozone cycle. Similarly, remote controls provide no mechanism for monitoring motion or occupancy within the treatment zone, resulting in a critical gap in safety protocols. These limitations highlight the need for systems that incorporate proactive, automated responses to ensure safe operation in real-time.

A key deficiency in current systems is the lack of integrated alert mechanisms to notify users or individuals entering the treatment area of potential hazards. Many devices do not provide real-time visual or audible alerts, leaving individuals unaware of ongoing ozone production or residual ozone levels. This omission is particularly problematic in environments with high foot traffic or shared use, where it is impractical to assume that all individuals are aware of the device's operational state. Consequently, the absence of robust safety alerts further increases the risk of unintended exposure.

Several prior art systems have attempted to address aspects of these safety concerns. For example, US2023347001A1 describes an advanced ozone sterilization system that uses motion detection sensors to detect human presence and automatically deactivate ozone generation when motion is detected. This system also includes real-time feedback mechanisms for alerting users to the device's operational status. However, it does not disclose sophisticated control over motion detection parameters, such as adjustable sensitivity, to reduce false triggers or tailor the system to specific settings. Additionally, it lacks multi-zone detection capabilities, which could enable monitoring of distinct areas simultaneously, a critical feature for larger or partitioned environments.

Similarly, CN212261975U discloses a portable ozone sterilizer with motion detection capabilities, utilizing sensors such as microwave radar or infrared. While this system incorporates a delay mechanism to confirm the absence of humans before resuming operation, it does not provide adjustable motion sensitivity to reduce false triggers in dynamic environments. Additionally, the system is confined to single-zone monitoring and does not address multi-zone configurations, leaving gaps in its ability to ensure comprehensive safety in complex or expansive spaces.

Other prior art systems, such as JP2002327937A, WO2019196015A1, and US2005207951A1, describe various implementations of motion detection and safety mechanisms for ozone generators. However, none of these systems disclose user-configurable motion sensitivity or the capability to monitor multiple zones simultaneously. These deficiencies limit their effectiveness in environments requiring precise and adaptable safety measures.

It is within this context of insufficient safety mechanisms, the absence of sophisticated motion detection control, and the lack of multi-zone safety configurations that the present invention is provided. This invention seeks to address these gaps by introducing innovative features such as adjustable motion sensitivity and multi-zone detection, alongside a manual restart mechanism, to provide enhanced safety, flexibility, and user control in ozone generator systems.

### SUMMARY

The present invention relates to an ozone generator system that incorporates a motion detection module, a control module, and an alert module to enhance safety during ozone generation. The motion detection module includes at least one motion sensor configured to monitor a designated area and detect the presence of motion. The control module is operatively coupled to the motion detection module and is configured to terminate ozone generation upon detecting motion and prevent further ozone generation until a manual restart command is received. The alert module, which includes an audible indicator and a visual indicator, is operatively coupled to the control module to provide real-time alerts in response to motion detection. An interface is provided for adjusting operational parameters of the system, including the sensitivity level of the motion detection module. The system further supports the use of multiple motion sensors to monitor distinct zones and manage ozone generation accordingly.

This configuration allows the system to address potential safety risks associated with ozone exposure by providing automated responses to motion detection and requiring deliberate user action to resume operation. The inclusion of adjustable motion sensitivity and multi-zone detection offers flexibility for deployment in a variety of environments, from residential spaces to large industrial areas, enhancing the practicality and safety of the system.

In some embodiments, the motion detection module includes a passive infrared (PIR) sensor, which provides a cost-effective and widely used solution for detecting motion based on changes in infrared radiation.

In further embodiments, the motion detection module includes alternative or additional sensors, such as millimeter-wave radar sensors, ultrasonic sensors, infrared time-of-flight sensors, or camera-based sensors, which improve detection accuracy and reduce false positives caused by environmental factors.

In yet further embodiments, the control module is configured to cross-validate signals from two or more motion sensors before terminating ozone generation, enhancing reliability and reducing false alarms.

In some embodiments, the alert module includes a piezoelectric buzzer and an LED indicator, providing clear and distinguishable alerts through sound and light.

In further embodiments, the audible indicator emits distinct patterns of sound signals during different stages of operation, such as intermittent signals during a delay period and continuous signals upon detection of motion, ensuring effective communication of the system's status.

In yet further embodiments, the visual indicator emits distinct colors to indicate different operational states, such as ozone generation, a delay period, or motion detection, providing an intuitive and immediate visual cue.

In some embodiments, the system includes a battery backup to ensure continued operation of safety features, such as motion detection and alerts, during power outages.

In further embodiments, the system includes an ozone concentration sensor operatively coupled to the control module, which monitors ozone levels and terminates ozone generation if a threshold concentration is exceeded, providing an additional safety layer.

In yet further embodiments, the interface allows users to adjust the motion detection sensitivity via a physical control, such as a rotary dial or slider, or through a digital touchscreen, offering adaptability for various environmental conditions.

In some embodiments, the interface is operatively coupled to a wireless communication module, enabling control and monitoring of the system via a mobile application, which also provides remote access to alerts and operational data.

In further embodiments, the motion detection module is positioned to minimize thermal interference from the ozone generation module, improving detection reliability in environments with fluctuating temperatures.

In yet further embodiments, the control module is configured to adjust motion detection sensitivity automatically based on environmental conditions, such as temperature or airflow, to reduce false triggers in dynamic settings.

In some embodiments, the motion detection module monitors overlapping zones to provide redundant safety coverage in high-risk areas, ensuring no motion events are missed.

In further embodiments, the control module delays the initiation of ozone generation for a pre-set period after receiving a start command, allowing sufficient time for users to vacate the area.

In yet further embodiments, the control module stores log data for detected motion events, including the time and specific zone, and displays this data through the interface, enabling users to review the system's operational history.

### BRIEF DESCRIPTION OF THE DRAWINGS

Various embodiments of the invention are disclosed in the following detailed description and accompanying drawings.

FIG. 1 illustrates an example system architecture of the ozone generator system, showing its components and their interconnections.

FIG. 2 illustrates an example flow diagram of the operational steps and decision-making processes of the ozone generator system.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The term “motion detection module” refers to any hardware or software component capable of detecting motion within a monitored area. This includes, but is not limited to, sensors such as passive infrared (PIR) sensors, millimeter-wave radar sensors, ultrasonic sensors, infrared time-of-flight sensors, or camera-based systems. In one example implementation, the motion detection module may include a PIR sensor configured to detect changes in infrared radiation emitted by objects within a specified range, while an alternative implementation may employ radar sensors to distinguish between stationary objects and moving individuals.

The term “control module” refers to any processing unit, microcontroller, or integrated circuit capable of receiving signals from the motion detection module, executing pre-programmed safety protocols, and controlling the operation of the ozone generation module. This includes, but is not limited to, microcontrollers such as ATmega328 or STM32, or custom-designed application-specific integrated circuits (ASICs). In one example implementation, the control module may be programmed to terminate ozone generation upon detecting motion and log the event for later review. Another example implementation may include a control module integrated with a wireless communication interface for remote monitoring and control.

The term “alert module” refers to any hardware or software component configured to provide real-time alerts in response to operational events, such as motion detection or ozone generation status changes. This includes, but is not limited to, audible indicators such as piezoelectric buzzers, visual indicators such as LEDs, or combinations thereof. In one example implementation, the alert module may emit a continuous sound signal and a flashing red light upon detecting motion, while another implementation may vary the light color based on operational states, such as green for active ozone generation and yellow for a delay period

The term “interface” refers to any hardware or software component that allows a user to interact with the system, adjust operational parameters, or monitor status. This includes, but is not limited to, physical dials, touchscreens, and mobile applications. In one example implementation, the interface may be a rotary dial on the device housing that allows users to adjust motion detection sensitivity. Alternatively, the interface may include a digital display and touch controls for setting delay times or monitoring system logs.

The term “motion detection sensitivity” refers to the capability of the motion detection module to respond to movements of varying intensity, range, or frequency. Adjustments to sensitivity may involve hardware modifications, such as tuning sensor thresholds, or software-based configurations through the control module. In one example implementation, sensitivity adjustments may be achieved using an adaptive algorithm that accounts for environmental conditions like temperature or airflow, while in another example, a user may manually adjust sensitivity through the interface to suit specific settings, such as reducing false triggers caused by small pets.

The term “multi-zone detection” refers to the ability of the motion detection module to monitor distinct areas or zones simultaneously, with each zone represented by one or more motion sensors. This includes configurations where zones are monitored independently or overlap for enhanced coverage. In one example implementation, a warehouse application may deploy multiple sensors positioned at strategic intervals, with the control module programmed to process signals from all zones and deactivate ozone generation if motion is detected in any zone. Another example implementation may involve a residential system with zone-specific alerts, such as identifying which room triggered the safety shutdown.

### Description of Drawings

The present invention relates to an ozone generator system designed to address the safety challenges associated with ozone generation, particularly the risks of unintentional exposure to humans or animals. Conventional ozone generators, while effective for air purification, odor removal, and disinfection, often rely on passive safety measures or basic automated systems that fail to respond dynamically to environmental changes. These limitations create significant risks, particularly in shared spaces or high-traffic environments where users may inadvertently enter areas during ozone generation or fail to deactivate the system after use.

This invention overcomes these shortcomings by integrating a motion detection module, a control module, and an alert module into the ozone generator system. The motion detection module continuously monitors the designated area for movement and communicates with the control module to terminate ozone generation immediately upon detecting motion. Unlike the prior art, the control module prevents automatic reactivation and requires a manual restart, ensuring deliberate user intervention before the system resumes operation. This approach mitigates the risks associated with unintentional or automatic reactivation, a critical gap in existing systems.

The invention further enhances safety and flexibility through features such as adjustable motion sensitivity and multi-zone detection. These improvements allow the system to be customized for various environments, reducing false triggers caused by minor movements, such as those from small pets or air currents, and enabling simultaneous monitoring of multiple zones in larger or partitioned spaces. These features address the lack of sophisticated motion detection controls and multi-zone configurations in prior art systems, which are typically limited to single-zone operation and static sensor parameters.

The inclusion of a dedicated alert module, comprising both audible and visual indicators, ensures real-time feedback to users when motion is detected or when the system is in operation. This integrated alert system replaces the reliance on passive warnings, such as labels or manuals, with proactive communication that is particularly beneficial in environments with multiple occupants or transient traffic.

By combining these features, the invention provides a robust, adaptive, and user-friendly solution for ensuring safety during ozone generation. It effectively bridges the gaps in current systems, offering enhanced functionality without compromising the core purpose of ozone-based air treatment.

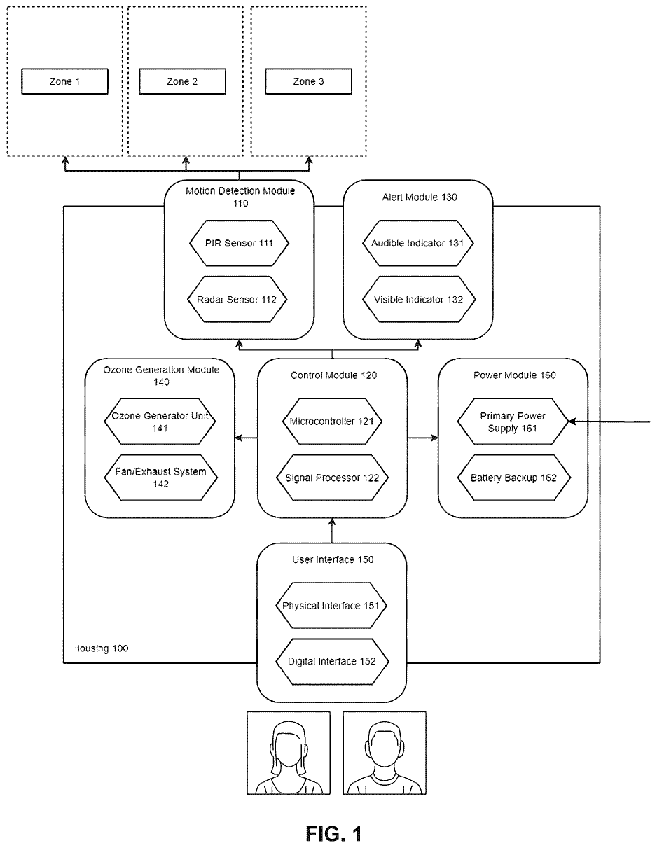

FIG. 1 illustrates a system architecture for an ozone generator system designed to enhance safety through motion detection, manual restart protocols, and configurable operational parameters. The system is housed within an enclosure 100, which may be formed from materials such as metal, plastic, or composite materials to provide structural integrity and thermal insulation. The housing 100 encloses and interconnects the primary functional modules, ensuring compact and efficient operation.

A motion detection module 110 is shown, comprising at least one motion sensor. In the example shown, the motion detection module 110 is depicted as including a passive infrared (PIR) sensor 111, configured to detect changes in infrared radiation caused by human or animal movement. In alternative implementations, the motion detection module 110 may include additional or alternative sensors, such as millimeter-wave radar sensors, ultrasonic sensors, infrared time-of-flight sensors, or camera-based sensors. These variants may offer enhanced precision or environmental adaptability, such as differentiating human presence from other heat sources or reducing false triggers caused by airflow or reflective surfaces.

The motion detection module 110 is operatively connected to a control module 120, which governs the operation of the system. The control module 120 is shown containing a microcontroller 121 and a signal processor 122. The microcontroller 121 processes input signals from the motion detection module 110 and executes safety protocols, such as terminating ozone generation upon motion detection and requiring manual restart before resuming operation. The control module 120 may be implemented using commercially available microcontrollers, such as an ATmega328 or STM32, or through custom-designed integrated circuits. The signal processor 122, in some embodiments, may include additional hardware or software components to support cross-validation of sensor inputs when the system uses multiple motion sensors.

An ozone generation module 140 is operatively coupled to the control module 120. The ozone generation module 140 includes an ozone generator unit 141 and a fan or exhaust system 142 for dispersing ozone into the monitored area. The ozone generator unit 141 may utilize technologies such as corona discharge or ultraviolet light to produce ozone. The fan 142 is designed to ensure even ozone distribution while minimizing thermal interference with the motion detection module 110, which may include shielding or placement adjustments as needed. In some implementations, the ozone generation module 140 may incorporate safety features such as an integrated ozone destructor to reduce residual ozone after operation.

The system further includes an alert module 130 operatively coupled to the control module 120. The alert module 130 is shown with an audible indicator, such as a piezoelectric buzzer 131, and a visual indicator, such as an LED 132. The audible indicator 131 emits distinct sound patterns depending on the operational state of the system, such as intermittent beeps during a delay period before ozone generation or continuous beeps when motion is detected. The visual indicator 132 emits different colors to represent various states, such as green for active ozone generation, yellow for a delay period, or red for a safety shutdown. In some embodiments, additional indicators such as LCD displays may supplement the alert module 130 to provide textual or graphical feedback.

An interface 150 is also shown, operatively connected to the control module 120. The interface 150 may include physical components, such as a rotary dial 151 or slider, for adjusting motion detection sensitivity or delay durations. Alternatively, the interface 150 may include a digital touchscreen 152, allowing users to configure additional parameters, such as ozone concentration thresholds or multi-zone detection boundaries. In some embodiments, the interface 150 may be connected to a wireless communication module, enabling remote monitoring and control through a mobile application.

A power module 160 is depicted, comprising a primary power supply 161 and a battery backup 162. The battery backup 162 ensures that critical safety functions, such as motion detection and alert systems, remain operational during power outages. In some implementations, the power module 160 may include energy-efficient components to extend battery life, particularly in environments where consistent mains power is unavailable.

The motion detection module 110 is further shown with a plurality of motion sensors arranged to monitor distinct zones, enabling multi-zone detection capabilities. The control module 120 processes signals from these sensors to determine the presence of motion in any zone and terminates ozone generation accordingly. In alternative embodiments, overlapping zones may be monitored to ensure no blind spots in high-risk areas. The system may also store data logs, including timestamps and detected zones, for later review through the interface 150.

Optional enhancements are not explicitly shown in FIG. 1 but are within the scope of the invention. These include an ozone concentration sensor operatively coupled to the control module 120 for real-time monitoring of ozone levels, with auto-shutdown if safe thresholds are exceeded. Additionally, adaptive algorithms may dynamically adjust motion detection sensitivity based on environmental factors, such as temperature or airflow, further reducing false triggers.

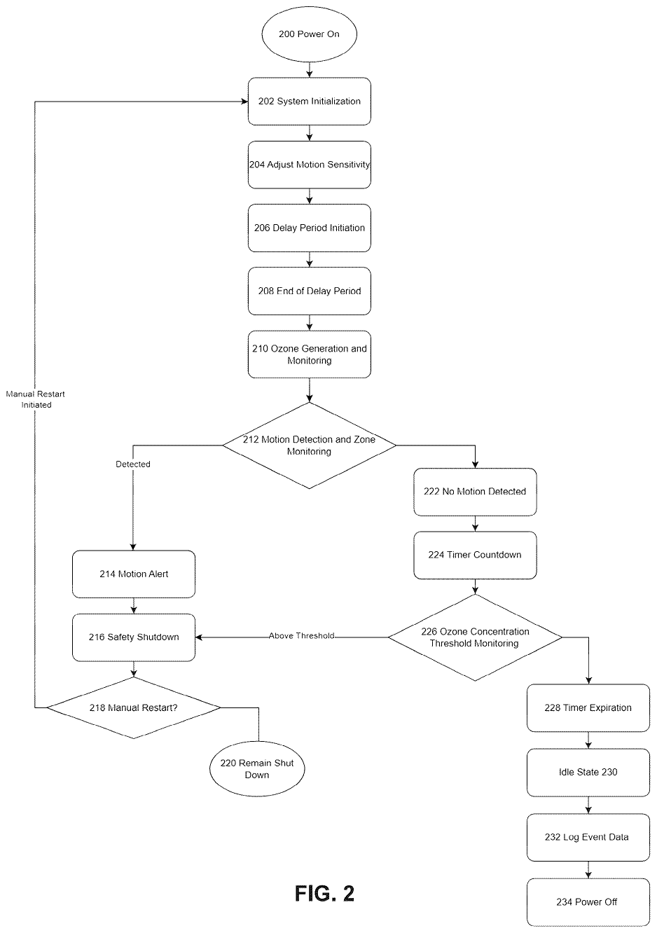

FIG. 2 illustrates a flow diagram depicting the operational sequence and decision-making processes of the ozone generator system. The system begins operation at step 200, where it is powered on by connecting it to an electrical supply. A power light illuminates to confirm that the system is receiving power. This initialization ensures that the control module and associated components are prepared for activation. The flow then proceeds to step 202, where the system enters an initialization phase. During this phase, the user activates the system via the interface, which may be a physical timer, touchscreen, or remote mobile application. The work indicator light turns on to signal that the system is active. At this stage, all modules, including the motion detection module, control module, ozone generation module, and alert module, are initialized and ready for operation.

In step 204, the user configures the motion detection sensitivity using the interface. This sensitivity adjustment can be implemented through various means, such as a physical dial, slider, or digital input on a touchscreen interface. In some implementations, the sensitivity may also be adjusted remotely via a mobile application connected through a wireless communication module. This configuration allows the user to tailor the system's responsiveness to the specific environment, such as reducing sensitivity in areas with small pets or drafts. In some embodiments, the control module may employ adaptive algorithms to dynamically adjust sensitivity based on environmental conditions, such as temperature or airflow, to further reduce false triggers.

The flow continues to step 206, where a delay period is initiated. The control module activates the fan and the audible indicator, which emits intermittent beeps at a user-configurable frequency, such as one beep per second. This delay period, which typically lasts one minute by default, provides time for the user to vacate the monitored area before ozone generation begins. The visual indicator may also emit a yellow light during this period to signify that the system is preparing for ozone generation.

Once the delay period ends at step 208, the audible indicator ceases, and the system transitions into active ozone generation mode at step 210. The control module activates the ozone generation module, which includes an ozone generator unit capable of producing ozone via technologies such as corona discharge or ultraviolet light. The fan ensures effective dispersion of ozone into the monitored area. Simultaneously, the motion detection module begins monitoring for motion within the designated zones. The motion detection module may include a single motion sensor, such as a passive infrared (PIR) sensor, or a combination of sensors, such as radar, ultrasonic, or camera-based sensors, to enhance detection accuracy.

At step 212, the control module evaluates signals from the motion detection module to determine if motion is detected. If the system is configured for multi-zone detection, the motion detection module processes inputs from multiple sensors placed in distinct zones. The control module assesses whether motion is detected in any zone, ensuring comprehensive monitoring coverage. If motion is detected, the system transitions to step 214, where the ozone generation module and fan are immediately deactivated. The alert module is activated, with the audible indicator emitting continuous beeps at a faster rate, such as one beep every 0.5 seconds, and the visual indicator flashing red. This combination of alerts ensures that individuals entering the area are warned of residual ozone presence.

Following motion detection, the system enters a safety shutdown state at step 216. During this state, the control module prevents further ozone generation until a manual restart command is received from the user. At step 218, the system awaits the manual restart command, which may be entered via the interface. If no restart command is received, the system remains in the safety shutdown state. If a restart command is received, the flow returns to step 202, where the system reinitializes.

If no motion is detected during the evaluation at step 212, the system continues to step 222, maintaining active ozone generation while monitoring the timer. At step 224, the timer begins counting down from the user-configured duration, which may range from one to 120 minutes. During this countdown, the control module also monitors ozone concentration levels using an ozone concentration sensor, as shown in step 226. If the ozone concentration exceeds a preset threshold, the control module triggers a safety shutdown by transitioning to step 214. If the ozone concentration remains within safe limits, the timer continues until it expires at step 228.

When the timer expires, the control module deactivates the ozone generation module and fan, and the system transitions to an idle state at step 230. In this state, the work indicator light turns off, and the system awaits further user commands. The flow concludes at step 234, where the user unplugs the system, deactivating all components.

Throughout the flow, the control module logs operational data at step 232, including motion detection events, ozone concentration levels, and shutdowns. This data may be accessed via the interface or a mobile application for review and analysis. The system's modular design allows for optional features such as adaptive sensitivity algorithms, zone-specific monitoring, and remote operation, enhancing its flexibility and suitability for various environments.

### Controller/Processor Components

A processor or controller as described herein may include any suitable type of computing device, such as a central processing unit (CPU), microcontroller, graphics processing unit (GPU), system on a chip (SoC), or digital signal processor (DSP). It may operate with one or more cores and may be configured to execute the functions described in this disclosure.

The processor may be operably connected to one or more memory devices, such as random access memory (RAM), read-only memory (ROM), flash storage, or solid-state drives (SSD). These memory devices store computer-readable instructions that, when executed by the processor, perform the methods described. The processor and memory communicate via data buses or other suitable communication pathways.

The computing device may also include input/output (I/O) devices, such as a touchscreen, mouse, keyboard, display, or speaker, to facilitate interaction with users or other systems. Additionally, it may include a network interface, such as a wired or wireless communication module, for connecting to networks.

Control logic or software instructions may be stored in memory and executed by the processor to implement specific functionalities. This logic may be modular, consisting of software components, processes, or functions that work together to perform the operations described herein.

The described computing operations involve the manipulation of data represented as electrical, optical, or magnetic signals stored or transferred within the system. These operations are machine-executed and do not require manual intervention, though they may interface with human operators through appropriate user interfaces.

The systems and methods described are not limited to any particular hardware configuration or programming language and may be implemented on general-purpose or specialized computing devices.

### Conclusion

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the ozone generator system of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An ozone generator system comprising:
a motion detection module including at least one motion sensor, the motion detection module configured to detect motion within a monitored area;
a control module operatively coupled to the motion detection module and an ozone generation module, the control module configured to:
initiate ozone generation in response to a start command;
monitor signals from the motion detection module to determine the presence of motion within the monitored area;
terminate ozone generation upon detection of motion within the monitored area; and
prevent further ozone generation until receipt of a manual restart command;
an alert module operatively coupled to the control module, the alert module including an audible indicator configured to emit a sound signal and a visual indicator configured to emit a light signal, both responsive to the detection of motion by the motion detection module;
an interface operatively coupled to the control module, the interface configured to receive input commands to adjust at least one operational parameter of the motion detection module, including a motion detection sensitivity level;
wherein the motion detection module comprises a plurality of motion sensors arranged to monitor distinct zones, and wherein the control module is configured to process signals from the motion detection module to manage ozone generation based on motion detected in any of the distinct zones.

2. The ozone generator system of claim 1, wherein the motion detection module includes at least one passive infrared (PIR) sensor.

3. The ozone generator system of claim 2, wherein the motion detection module further includes at least one alternative motion sensor selected from the group consisting of millimeter-wave radar sensors, ultrasonic sensors, infrared time-of-flight (ToF) sensors, and camera-based sensors.

4. The ozone generator system of claim 1, wherein the control module is configured to verify the presence of motion in the monitored area by cross-validating signals from two or more motion sensors before terminating ozone generation.

5. The ozone generator system of claim 1, wherein the control module is further configured to determine the specific zone in which motion is detected and generate zone-specific alert signals through the alert module.

6. The ozone generator system of claim 1, wherein the alert module includes a piezoelectric buzzer as the audible indicator and an LED as the visual indicator.

7. The ozone generator system of claim 6, wherein the audible indicator emits a first pattern of intermittent sound signals during a delay period prior to ozone generation and a second pattern of continuous sound signals upon detection of motion.

8. The ozone generator system of claim 6, wherein the visual indicator emits a first color signal during ozone generation, a second color signal during a delay period prior to ozone generation, and a third color signal upon detection of motion.

9. The ozone generator system of claim 1, further comprising a battery backup configured to power the motion detection module, the control module, and the alert module during power outages.

10. The ozone generator system of claim 1, further comprising an ozone concentration sensor operatively coupled to the control module, wherein the control module is configured to terminate ozone generation if an ozone concentration threshold is exceeded.

11. The ozone generator system of claim 10, wherein the control module is further configured to display real-time ozone concentration levels through the interface.

12. The ozone generator system of claim 1, wherein the interface includes a physical rotary dial, slider, or touchscreen for adjusting the motion detection sensitivity level.

13. The ozone generator system of claim 1, wherein the interface is operatively coupled to a wireless communication module configured to enable control of the system via a mobile application.

14. The ozone generator system of claim 13, wherein the mobile application is configured to receive alerts from the alert module and allow users to adjust operational parameters of the control module.

15. The ozone generator system of claim 1, wherein the motion detection module is positioned to minimize thermal interference from the ozone generation module.

16. The ozone generator system of claim 1, wherein the control module is configured to adjust the motion detection sensitivity automatically based on environmental factors, including ambient temperature or airflow conditions.

17. The ozone generator system of claim 1, wherein the motion detection module is configured to operate in overlapping zones to provide redundant monitoring of high-risk areas.

18. The ozone generator system of claim 1, wherein the control module is further configured to delay the initiation of ozone generation for a pre-set period after receiving the start command, the delay period being adjustable through the interface.

19. The ozone generator system of claim 1, wherein the control module is configured to store log data for each detected motion event, including the time and specific zone, and display this data through the interface.

20. The ozone generator system of claim 1, wherein the plurality of motion sensors in the motion detection module are wirelessly connected to the control module.
